(19) United States
(12) Patent Application Publication
(10) Pub. No.: US 2004/0203069 A1
(43) Pub. Date: Oct. 14, 2004

(54) Method for determining and/or isolating lipid-binding compounds

(76) Inventor: Chris Reutelingsperger, Maastricht (NL)

Correspondence Address: ANTHONY H. HANDAL, KIRKPATRICK & LOCKHART, LLP, 599 LEXINGTON AVENUE, 31ST FLOOR, NEW YORK, NY, 10022-6030, US

(21) Appl. No.: 10/410,684

(22) Filed: Apr. 10, 2003

(51) Int. Cl. G01N 33/53

(52) U.S. Cl. 435/7.1

(57) ABSTRACT

The invention relates to a method for determining rapidly the biological activity of lipid-binding compounds, such as Annexins. The compounds may be are labeled. The labeled compounds are mixed with divalent cations and with a, particulate or other, carrier containing negatively charged phospholipids. The mixture is incubated and, subsequently, submitted to a procedure to separate the carrier from the solution. This separation procedure can involve for example a magnet, if (para)magnetic particles are used, a centrifugation step or a filtration through a filter, the pores of which are smaller than the particles. The distribution of label over the solution and the carrier reflects the portion of biologically active labeled compound. The presence of neutralizing factors in a sample can be measured by contacting the sample with a known amount of phospholipids on the career.

### FIELD OF THE INVENTION

The present invention relates to the field of medical analysis. More particularly, it relates to diagnostic analyses to determine cell death in patients, such as cancer and cardiovascular patients, using labeled detecting reagents, such as annexins. There is a need to measure the quality of labeled annexin quickly after the labeling before it will be injected into patients. Currently, the radiopharmacists and the nuclear clinicians and technicians can only determine the physical yield of radiolabeled annexin. The present invention provides a rapid method for determining also the biological activity of labeled annexin. At the same time, the invention provides methods of determining the presence of neutralizing compounds in biological environments that inhibit the lipid binding of reagents such as annexins,

### BACKGROUND

Programmed cell death (PCD) is mandatory to normal development and tissue homeostasis of the adult organism. It is characterized by a sequence of morphological and biochemical changes (Leist and Jattela, 2001). Derailment of PCD is believed to be part of the etiology and pathology of congenital malformations, cardiovascular diseases, neurodegeneration and cancer. Instead of looking at PCD as being a cause of disease, it may also be employed as a target in the clinic to diagnose and treat diseases. For instance, many anti-cancer therapies such as chemo- and radiotherapy are effective because they induce PCD of the tumor cells (Kerr et al., 1994). Hence, the measurement of PCD in vivo in patients provides information about the pathology and the efficacy of therapy,

Recently, a procedure was developed to measure PCD in patients non invasively with radionuclide imaging (Hofstra et al. 2000, Hofstra et al. 2001, Narula et al. 2001). This procedure is based on the experimental findings that cells executing PCD expose phosphatidylserine (PtdSer) at their surface (Fadok et al. 1992). While living cells generate and maintain PtdSer localized in the inner plasma membrane (Zwaal and Schroit 1997) facing the cytosol, dying cells translocate PtdSer to the outer plasma membrane leaflet for signaling their suicide to the environment (Padok et al. 1992). The procedure is also based on the phospholipid binding properties of Annexin A5 (AnxA5), which is a member of the Annexin family (Gerke and Moss 2002). AnxA5 binds specifically to phospholipid surfaces, which comprise negatively charged phospholipids such as PtdSer (Tait et al. 1989, U.S. Pat. No. 5,258,497, Stuart et al. 1998). AnxA5 binding to these phospholipid surfaces is dependent on the presence of calcium ions and is reversible, Chelation of calcium ions by agents such as ethylenediamine-tetraacetic acid (EDTA) prevents phospholipid binding of AnxA5 and causes bound AnxA5 to detach from the phospholipid surface (Andree et al. 1990). This phospholipid binding property forms the basis of its ability to detect PCD (U.S. Pat. No. 5,834,196) in vitro (van Engeland et al. 1998) and in vivo (van den Eijnde et al. 1997). M1234, a mutant of AnxA5 which lacks the ability to bind to PtdSer (Mira et al. 1997), does not bind to dying cells in vivo (Dumont et al. 2000, Dumont et al. 2001).

The above-mentioned procedure of radionuclide imaging of PCD in vivo employs radiolabeled AnxA5 (U.S. Pat. No. 6,197,278). It was firstly validated in animal models (Blankenburg et al, 1998) and, secondly, successfully applied in patients using Techetium-99m (^{99m}Tc)-labeled AnxA5 and SPECT (Single Photon Emission Computed Tomography) analysis (Hofstra et al. 2000, Hofstra et al. 2001, Narula et al. 2001). The clinical procedure involves the radiolabeling of AnxA5 prior to injection into the patient. Most of the clinically applied radionuclides have a short half-life time such as ^{99m}TC (t_{1/2}=±6 h), ¹⁸F (t_{1/2}=±1.8 h) and ¹¹C (t_{1/2}=±20 min), These short half-lifes set a narrow time-window between radiolabeling and injection into the patient for analysis of the radiolabeled AnxA5. To date it is general practice to analyse only the labeling efficiency of the procedure in terms of % of radiolabel associated with AaxA5. The results of such physical analysis do not provide any information about the biological activity, and consequently, the PCD imaging capacity of the radiolabeled AnxA5. On the other hand, the blood of the patient may contain compounds that inhibit the biological activity of radiolabeled AnxA5, and that may cause false negative results. Consequently, the PCD imaging potency of the radiolabeled AnxA5 may become hampered by the specific biology of the patient,

Hence, there is a great need for a rapid and simple method for determining the biological activity of labeled lipid-binding proteins such as AnxA5, which are prepared for clinical use and for determining the presence of AnxA5-neutralizing compounds in the blood of the patient. The present invention provides in such need,

### DETAILED DESCRIPTION OF THE INVENTION

The invention provides a method for determining the biologically active fraction from a mixture of lipid binding and optionally also non-lipid binding compounds making use of:

a: a solid carrier carrying lipids, and

b: a separator to separate the lipid-carrying carrier from the liquid, and

c: divalent cations.

The term “biologically active” as used herein in the context of lipid-binding compounds, means having a biologically effective lipid-binding property, where appropriate taking into account the presence of inhibiting and promoting factors and conditions. In the method of the invention the biologically active, lipid-binding compounds can be labeled. The labeled compounds are mixed with the carrier which contains the lipids. Where lipid binding is dependent on to the presence of cofactors, such as divalent cations, such cofactors, e.g. Ca²⁺ ions are admixed as well. The mixture is incubated and, subsequently, submitted to a procedure to separate the carrier from the liquid. This separation procedure can involve for example a magnet, if the particles are (para)magnetic, a centrifugation step, or a filtration step using a filter, the pores of which are smaller than the particles. The amount of the label on the particles or the distribution of the label over the liquid and the carrier reflects the portion of biologically active labeled compound.

The solid carrier may be a carrier in the form of a container wall, a filter, or a packing of a container or column. Preferably, however, the solid carrier consists of particles, e.g. granules, beads, rods, microspheres or the like having spherical, elliptic or other shapes. The particles may have a particle size of less than 1 μm tot several nun, e.g. from 1 μm to 10 mm, especially from 5 μm to 1 mm. In an advantageous embodiment, the particles may contain metal and/or metal oxides. The metals and metal oxides may serve to increase density of the particles and thus to enhance the separability of the particles using gravity. The metals may, instead or in addition, have or be susceptible to induction of magnetic properties, such as iron or nickel, thus improving separability using magnetic forces. Thus, separation of the particles from the liquid of the sample can be effected using a permanent magnet or an inducible magnet as the separator. The solid carrier prepared from metal or metal oxides may be combined with organic materials such as polymers. Suitable examples of polymer materials for use in combination with metal or metal oxides are agarose, cellulose, dextran or other poorly soluble or non-soluble polysaccharides, collagen or other poorly soluble proteins, poly-acrylamide, polyvinyl chloride, polyvinyl alcohol, polyethyleneglycol, polystyrene and latex. Such polymers may be crosslinked to enhance structural strength and water resistance, if necessary,

The separator may thus be a permanent magnet or a inducible magnet. Alternatively, the separator may be a filter with pores, which are smaller than the described above. Thus, depending on the particle size, the pore size may be e.g. from 0.1 μm to 1 mm. As a variant thereof, the separator can be a container containing and retaining the particles described above and having small outlets, with sizes smaller than the particle size, for bringing the liquid of the sample in and out of the container.

When the particles, together with the lipids, have a density well below or well above the density of the liquid, (e.g. a density below 0.95 g/cm³ or above 1.1 g/cm³ in case of aqueous liquids), the separator may be a device separating by gravity, e.g. a centrifuge or a decanter.

For the purpose of detecting or isolating biological material binding to only specific lipids, these specific lipids should be present on the carrier, optionally in admixture with other lipids. The specific lipid should preferably account for at least 1%, more preferably at least 10% of the total lipid coating. In case of detecting or isolating proteins of the annexin type as described below, the specific lipid should be negatively charged, such as phosphatidyl-serine, phosphatidyl-inositol, phosphatidic acid, cardio-lipin, their lyso analogs and the like. Vice versa, the lipids may comprise phospholipids having cationic groups bound to the phosphatidyl group, such as phosphatidyl-choline, phosphatidyl-ethanolamine and the like for specifically detecting or isolating the corresponding biological materials.

The lipid applied on the solid carrier can be any lipid capable of being bound by a biological material, especially by lipid-specific proteins as described below. Preferred lipid are phospholipids, i.e. lipids having a phosphatidyl moiety. Depending on the lipid-binding biological material, the phospholipids may be of the type having acidic or anionic functions such as phosphoric or carboxyl acid functions, neutral functions such as in sugar-derived phospholipids, or basic or cationic functions such as amine and ammonium functions., Other lipids, including triglycerides, but also lipophilic materials such as cholesterol and the like may also be present on the solid carrier. Mixtures of lipids are also suitable. Preferably, a lipid mixture which resembles the lipid composition of biological membranes (Lehninger, 2000), i.e. containing different phospholipids, such as anionic (e.g. phosphatidyl serine, cardiolipin and phosphatidic acid) and neutral (e.g. phosphatidyl choline and phosphatidyl ethanolamine) phospholipids, in association with other lipids, optionally in combination with terpene-based lipids such as sterols, is applied on the solid carrier.

The phospholipids may be of the fully esterified type, i.e. a glycerol moiety carrying one phosphatidyl function and two fatty acid chains. Examples thereof include phosphatidylserine, phosphatidic acid, phosphatidylinositol, phosphatidylethanolamine, phosphatidylcholine and sphingomyelin. Analogs thereof of the diphosphatidylglycerol type such as cardiolipin, or of the type wherein glycerol is replaced by a partially or fully esterified other polyol, such as glycol, erythritol, glucose or sorbitol are also suitable. The phospholipids may also be of the partially esterified type, i.e. a glycerol moiety carrying one phosphatidyl function and only one fatty acid chain; these are commonly referred to as “lyso-” derivatives. Suitable examples include lysophosphatidylserine, lysocardiolipid, lysophosphatidic acid, lysophosphatidylinositol, lysophosphatidyl-ethanolamine and lysophosphatidylcholine,

The lipids are preferably present on the solid carrier as a lipid coating of several nm (monolayer) up to e.g. 1 μm thickness. The lipids may be applied e.g. by mixing the carrier with the lipid in a suitable solvent, followed by evaporation of the solvent, as described by Stuart et al., 1998. Alternatively, the lipid may be covalently bound to the carrier using common coupling reactions depending on the nature of the carrier. For example, a carrier containing protein material can be covalently bound to a lipid using amine functions of the protein and aldehyde functions introduced on the lipid, e.g. using phosplatidyl aldelhydes, or a carrier containing polysaccharide material, e.g. dextran, can be covalently bound using aldehyde functions on the polysaccharide or introduced amino functions on the polysaccharide, or other art-known coupling reagents. The amount of lipid on the carrier may be e.g. 0.1-100, especially 0.5-50 nmoles lipid per cm² of carrier surface or e.g. 0.07-70, especially 0.35-35 μg lipid per cm² of carrier surface. Alternatively, the amount of lipid may e.g. 10-10000, especially 50-5000 μmol lipid per g of carrier material, or e.g. 8-8000, especially 40-4000 mg lipid per g of carrier material.

The biological material can be any compound or mixture capable of binding to lipids or lipid fractions. Herein, “binding” does not necessarily mean binding through chemical bonds it may also be through physical binding, as long as there is sufficient adherence to overcome the solvation energy in the liquid of the sample. Preferably, the dissociation constant of the compound to the lipid K_{D}=[A].[L]/[AL], wherein [A] and [L] are the molar concentrations of the lipid-binding compound and the lipid, respectively, and [AL] is the Molar concentration of bound complex of A and L, is below 10⁻⁵ M, most preferably 10⁻⁶ M or lower. The compound can advantageously be a polypeptide. A particularly interesting group of polypeptides are the proteins that different binding characteristics with different types of lipids. Examples are compounds capable having different affinities to unsaturated lipids vs. saturated lipids, or having different affinities to apolar lipids, such as triglycerides, vs. more polar lipids, such as glycerol derivatives having only one or two fatty acid groups and one or two polar groups, for example phospholipids, glycolipids and the like.

Particularly interesting are compounds, especially polypeptides having specific affinities for lipids having neutral or anionic functions, especially phosphatidylserine and similar lipids. Such compounds include members of the annexin family described above. Several of such members of the annexin family can be used, such as Annexin A5 and Annexin A8. Annexin A5 (formerly referred to as VACα) is especially preferred. Derivatives of annexins, such as annexins having a mutated or truncated amino acid sequence, or being bound to other peptide sequences or non-peptide fragments, can also be used.

Where the lipid-binding compounds are used as analytes, they may suitably be provided with a label allowing their detection and optionally isolation after the lipid-binding process. Suitable labels are commonly known in the art, such as fluorescence labels, radionuclides, dyes, enzyme labels, and the like. A preferred groups of labels are radioactive tracers, such as ^{99m}Tc, ¹²³I, ¹²⁴I, ⁶⁷Ga, ¹¹¹In, ¹¹C, ¹³N, ¹⁵O, ¹⁸F, ⁶⁸Ga, ⁸²Rb, ⁸⁹Zr. Useful fluorescent probes can be selected from fluorescein, FITC, APC, PerCP, GFP, RPP, Texas Red, phycoerytlrin, rhodamine, carboxytetramethylrhodamine, DAPI, indopyra dyes, Cascade blue coumahin, NBD, Lucifer Yellow, propidium iodide, a porphyrin, BODIPY, CY.sup.3, CY sup.5, CY.sup.9, and derivatives and analogs thereof. Another useful label is all antibody or part thereof that can be bound to the compound, and allows detection by contacting it with the specific antigen in a manner known in the art for immunoassays.

The binding of various lipid-binding materials is dependent on the presence of divalent cations such as calcium, magnesium, zinc, strontium etc. Therefore these metals are used when determining such materials. Where lipid-binding compounds do not require metal assistance in binding, or require other agents, cofactors, higher-valent metals or the like, such as anti-phospholipid binding antibodies, β2-glycoprotein I and cytochrome C, the method can be carried without such metals or with such other agents.

The sample to be used in the method of the invention can be any material containing or suspected of containing one or more lipid-binding compounds. The sample may be a biological sample taken from a human or animal or even vegetal or microbial subject, possibly after further pre-treatment, or it may be a synthetic sample, e.g. prepared reagent which needs testing on effectivity. The sample may be from liquid or solid origin. If it is solid, it is preferably liquefied by addition of a solvent or diluent, usually buffered water or aqueous mixtures, where necessary using dissolution aids, stabilizers etc. After further dilution as necessary, the sample can be subjected to the lipid-binding assay as described above and further illustrated in examples below.

The method of the invention is especially useful for determining the presence of factors, in biological media, that are capable of neutralizing the biological activity of lipid-binding compounds, in particular of neutralizing the phosphatidyl-serine-binding capacity of annexins, such as antibodies against the lipid-binding compounds. Such factors or compounds may be present in blood or plasma of patients in which the lipid-binding activity is to be determined. Thus, a sample of such medium e.g. a blood or, preferably, plasma sample is contacted with a known amount of lipid-binding compound such as annexin, preferably labeled, before contacting the lipid-binding compound with the solid carrier carrying the lipids. Also less preferred, the admixing of the biological sample can also be done to the carrier already carrying the lipids, or simultaneous with contacting the carrier with the lipid-binding compound. The neutralizing activity in the sample can then be assayed from the degree of reduction of specific lipid-binding as found,

The invention also pertains to a kit for performing the method described above. The kit contains a solid carrier having attached thereto a lipid, and additionally further reagents, diluents, detections aids and like. A particular reagent to be used in the kit is a divalent cation such as magnesium, calcium, zinc, etc. especially calcium. The divalent cation is especially present as an aqueous solution, e.g. containing 0.1-10 mM of divalent cation salt, optionally together with buffering compounds, metal complexants, saline etc. The kit may also contain a separator allowing separation of the carrier after the lipid-binding reaction such as a filter, a container, or a magnet.

### EXAMPLE 1

In a particular embodiment of the invention paramagnetic particles of iron oxide in a polyvinylalcohol matrix are coated with a lipid mixture of phosphatidylcholine (PtdCh) and phosphatidylserine (PtdSer). In this example the lipid mixture is composed of equimolar quantities of PtdCh and PtdSer. The embodiments of this invention include also particles containing other lipid compositions if a negatively charged phospholipid is present.

1 μmole PtdCh in CHCl₃ and 1 μmole PtdSer in CHCl₃ are mixed in a glass container and dried under nitrogen to give a film of lipids on the glass wall, 50 mg unmodified M-PVA type 2 paramagnetic beads (Chemagen, Del.) are added to the glass container and vortexed over the lipid film. The particles will take up the lipids and form a lipid coat on their surface. A monolayer of phospholipids on the surface of the particles comprises approximately 0.25 nmoles lipid per cm², a bilayer about 0.5 nmoles lipid per cm² etc. The particle-lipid suspension is ultrasonicated with an amplitude of 6μ (Soniprep 150) during 60 s. The coated particles (PtdCh/PtdSer particles) are washed with an aqueous buffer to remove unbound lipid liposomes. The washed PtdCh/PtdSer particles are resuspended in buffer containing 25 mM Hepes/NaOH, ph 7.4, 140 mM NaCl, and 2.5 mM CaCl₂ (Ca²⁺ buffer) or 25 mM Hepes/NaOH, pH 7.4, 140 mM NaCl, and 1 mM EDTA (EDTA buffer). The embodiments of this invention are not limited to these buffer compositions. For example, the Ca²⁺ buffer can contain other concentrations of Ca²⁺ ions providing that the chosen Ca²⁺ ion concentration promotes binding of AnxA5 to the lipid coated particles. The EDTA-buffer is chosen because it forms a negative control to determine background binding. Any other buffer, which does not promote divalent cation dependent binding of AnxA5 to the lipid coated particles can be used instead.

The AnxA5 binding properties of the PtdCh/PtdSer particles are analysed with fluorescein-labeled AnxA5 (AnxA5-FITC, NeXins Research BV, the Netherlands) using light microscopy and flow cytometry.

PtdCh/PtdSer particles in Ca²⁺ buffer are incubated with 250 ng/ml AnxA5 for 5 minutes or more and subsequently visualized with light and fluorescence microscopy. These visualization procedures clearly illustrate that the PtdCh/PtdSer particles bind AnxA5-FITC.

Next, PtdCb/PtdSer particles in Ca²⁺ buffer or EDTA buffer are incubated with 250 ng/ml AnxA5 for 5 minutes and the particles are analyzed for bound Annexin A5-FITC by flow cytometry. The flow cytometric analysis demonstrates that the PtdCh/PtdSer particles bind AnxA5-FITC in Ca²⁺-dependent manner. In order to demonstrate that the nature of this binding concerns the specific interaction between AnxA5 and PtdSer, paramagnetic particles are prepared with a PtdCh coat only. Incubation of the PtdCh particles with AnxA5-FITC in the presence of Ca²⁺ ions does not result in the binding of AnxA5-FITC to the PtdCh particles, indicating that PtdSer is required for AnxA5-FITC binding. On the other hand, PtdCh/PtdSer particles are incubated with the AnxA5 mutant M1234FITC, the four Ca²⁺/phospholipid binding sites of which are inactivated by the replacement of four amino acids (Mira et al. 1997). M1234-FITC does not bind to PtdCh/PtdSer particles in the presence of Ca²⁺ ions, indicating functional Ca²+/−phospholipid binding sites of AnxA5 are required for binding to PtdCb/PtdSer particles.

### EXAMPLE 2

An embodiment if this invention involves the separation of lipid coated particles from the solution in order to determine the content of biologically active labeled 5.

1 μmoles PtdCh in CHCl₃, 1 μmoles PtdSer in CHCl₃ and 1 μmoles cholesterol (Chol) are mixed in a glass container and dried under nitrogen to give a film of lipids on the glass wall. 50 mg unmodified M-PVA type 2 paramagnetic beads (Chemagen, Del.) are added to the glass container and vortexed over the lipid film. The particles will take up the lipids and form a lipid coat on their surface. A monolayer of phospholipids on the surface of the particles comprises approximately 0.25 nmoles lipid per cm², a bilayer about 0.5 nmoles lipid per cm² etc. The particle-lipid suspension is ultrasonicated with an amplitude of 6μ (Soniprep 150) during 60 s. The coated particles (PtdCh/PtdSer/Chol particles) are washed with an aqueous buffer to remove unbound lipid liposomes. The washed PtdCh/PtdSer/Chol particles are resuspended in buffer containing 25 mM Hepes/NaOH, pH 7.4, 140 mM NaCl, and 2.5 mM CaCl₂ (Ca²⁺-buffer) or 25 mM Hepes/NaOH, pH 7.4, 140 mM NaCl, and 1 mM EDTA (EDTA-buffer).

Various amounts of PtdCh/PtdSer/Chol particles are mixed with 1 μg fluorescein-labeled AnxA5 (AnxA5-FITC, NeXins Research BV, NL) and incubated for 5 minutes or more. The vial containing the mixture is then placed in a magnet, which causes the attraction of the PtdCh/PtdSer/Chol particles towards the magnet and the assembly of the particles as a dark brown pellet at the vial's wall adjacent to the magnet. Thus, the solution containing the unbound AnxA5-FITC can be separated from the particles containing the bound AnxA5-FITC. The AnxA5-FITC concentration in the solution is measured by fluorimetry. The results show that the AnxA5-FITC can be depleted from the solution for more than 95% by the PtdCb/PtdSer/Chol particles in the presence of Ca²⁺ ions. In the presence of EDTA no depletion is observed. The Ca²⁺-dependent depletion depends on the intact Ca²⁺/phospholipid binding sites of AnxA5 since M1234-FITC is not depleted from the solution in this system. Preincubation of AnxA5-FITC with the anti-AnxA5-specific monoclonal antibody WAC2a (Tau Technologies) abolishes the binding of AnxA5-FITC to the particles, Talcen together this demonstrates that depletion by the particles reflects the biological activity of AnxA5.

Identical results are obtained if the separation of the particles from the solution occurs through centrifugation and filtration for example through a 0.22 μl filter (Sterivex-GV, Millipore or Costar).

### EXAMPLE 3

An embodiment of this invention is the determination of the contents of biologically active radiolabeled AnxA5 by mixing radiolabeled AnxA5 with lipid coated particles in the presence of Ca²⁺ ions or EDTA and, subsequently, separating the particles from the solution.

Hynic-^{99m}Tc-AnxA5 is prepared with the Apomate™ kit from Thesens Imaging Inc. according to the protocol of the manufacturer. An aliquot of the Hynic-^{99m}Tc-AnxA5 solution is withdrawn and added to a vial containing 3 mg/ml PtdCh/PtdSer/Chol beads in Ca²⁺ buffer or EDTA buffer obtained according to example 2. The suspensions are mixed and incubated for 5 minutes or more. Then, the vials are placed in the magnet to separate the particles from the solution. This separation can also be accomplished by centrifugation or by filtrating for example through a 0.22μ filter (Sterivex-GV, Millipore). The separated solution and particles are measured separately by scintillation counting. Typical results are presented in table 1. The results are transposed in % biologically active Hynic-^{99m}-Tc-AnxA5 by the formula:

$$\frac{( {S}_{EDTA} - {S}_{Ca} 2 + ) * 100 \%}{{S}_{EDTA}}$$

where S_{EDTA}=the radioactivity in the solution if EDTA-buffer is used

and S_{Ca}2+=the radioactivity in the solution if Ca2+-buffer is used. In this example the % biologically active Hynic-^{99m}Tc-AnxA5 is 96%

TABLE 1

| | counts in particles | counts in solution |
|---|---|---|
| Buffer | (μCi) | (μCi) |
| Ca²⁺-buffer | 1789 | 61 |
| EDTA-buffer | 59 | 1732 |

### EXAMPLE 4

In a particular embodiment of the invention lipid coated particles are employed to determine the presence of compounds in the blood of human that neutralize the biological activity of labeled AnxA5. The biopotency of labeled AnxA5, e.g. the potency to bind to apoptotic cells in the complex environment of the human organism, can be diminished by compounds in the blood that prevent labeled AnxA5 from binding to phospholipid surfaces with negatively charged phospholipids such as PtdSer. The current invention allows rapid assessment of the presence of AnxA5-neutralizing compounds in the blood of human. In this example the lipid mixture to coat particles mimicks the lipid composition of biological membranes as given in the textbook Lehninger, Principles of Biochemistry, third edition 2000, by David L. Nelson and Michael M. Cox on page 393.

1.82 μmoles L-α-phosphatidylcholine (egg, Avanti Polar Lipids), 0.49 μmoles L-α-phosphatidylserine (brain, Avanti Polar lipids), 0.98 μmoles L-α-phosphatidylethanol-amine (heart, Avanti Polar lipids) and 1.7 μmoles cholesterol (Avanti Polar lipids) are mixed in a glass container. The solvent CHCl₃ is evaporated under nitrogen to yield a film of dried lipids on the glass wall. 1 ml of 5 mg/ml unmodified M-PVA type 2 paramagnetic beads (Chemagen, Germany) is added to the glass container. The solution, which covers fully the lipid film, is vortexed over the film for 5 minutes, The particles will take up the lipids and form a lipid coat on their surface. A monolayer of phospholipids on the surface of the particles comprises approximately 0.25 nmoles lipid per cm², a bilayer about 0.5 nmoles lipid per cm² etcetera. The particle-lipid suspension is ultra-sonicated with an amplitude of 6μ (Soniprep 150) during 60 s. The coated particles (PtdCh/PtdEth/PtdSer/Chol particles) are washed with an aqueous buffer to remove unbound liposomes. The washed PtdCh/PtdEth/PtdSer/Chol particles are resuspended in buffer containing 25 mM Hepes/NaOH, pH 7.4, 140 mM NaCl, 1 mg/ml BSA and 1 mM EDTA (EDTA-buffer).

A fixed amount of PtdCh/PtdEth/PtdSer/Chol particles is mixed with 10 ug/ml fluorescein-labeled AuxA5 (AnxA5-FITC, NeXins Research BV, the Netherlands) and incubated for 5 minutes or more in Ca²⁺ buffer (25 mM Hepes/NaOH, pH 7.4, 140 mM NaCl, 1 mg/ml BSA, and 2.5 mM CaCl₂). Then the particle suspension is analysed for the amount of bound AnxA5-FITC per particle by flow cytometry. These analyses determine the reference point for 100% biologically active AnxA5-FITC or the reference point for the situation without the presence of compounds that neutralize the biological activity of AnxA5. Human serum or plasma samples can be analysed on the presence of labeled AnxA5-neutralizing compounds by mixing the sample with for example 10 ng/ml AnxA5-FITC and adjusting the mixture to Ca²⁺>0.1 mM, if necessary. A fixed amount of PtdCh/PtdEth/PtdSer/Chol particles (the same amount as with which the reference point is prepared) is added to the mixture and incubated for 5 minutes or more. Then the particle suspension is analysed for the amount of bound AnxA5-FITC per particle by flow cytometry. The amount of bound AnxA5-FITC relative to the reference point reflects the presence and potency of labeled AnxA5-neutralizing compound(s) in the blood sample.

The presence and potency of labeled AnxA5-neutralizing compound(s) in blood samples can also be assessed by using AnxA5 labeled with other fluorescent groups, biotin, antibodies or fragments thereof, and radionuclides and separators such as magnets, filters and centrifuges

### EXAMPLE 5

In a particular embodiment of the invention the blood of a patient is analyzed on the presence of compounds that neutralize the biological activity of Hynic-^{99m}Tc-AnxA5. Such compounds can for example be antibodies against Hynic-^{99m}Tc-AnxA5. These neutralizing compounds can cause false negative results if the patient would receive Hynic-^{99m}Tc-AnxA5 for imaging of PCD. Hence, the results of this particular embodiment are important to clinical decision-making.

PtdCh/PtdEth/PtdSer/Chol-particles are prepared as described above. Blood is withdrawn from the patient with informed consent and anticoagulated with for example heparin. Plasma is prepared by centrifugation according general practice. Hynic-^{99m}Tc-AnxA5 is prepared with the Apomate™ kit from Theseus Imaging Inc. according to the protocol of the manufacturer. An aliqout of Hynic-^{99m}Tc-AnxA5 is mixed with Ca²⁺ buffer or the patient's plasma After an incubation time an aliquot of PtdCh/PtdEth-PtdSer/Chol particles is added to the mixtures and the resulting suspension is further incubated. The suspension is then placed in a magnet to separate the particles from the solution. The solution is analyzed by scintillation counting. The differences in counts between the Hynic-^{99m}Tc-AnxA5 incubation in buffer and in the patient's plasma indicates the presence and potency of compounds neutralizing the biological activity of Hynic-^{99m}Tc-AnxA5 in the plasma of the patient.

List of Abbreviations

Abbreviation Term

AnxA5 Annexin A5

Apomate™ Hynic-Annexin A5

Chol Cholesterol

EDTA Ethylenediamine-tetraacetic acid

Hynic Hydrazineonicotinamide

PCD Programmed Cell Death

PtdCh Phospliatidylcholine

PtdEth Phosphatidylethanolamine

PtdSer Phosphatidylserine

99 mTc Technetium-99m

References Cited

Andree, H. A., Reutelingsperger, C. P., et al., “Binding of vascular anticoagulant alpha (VAC alpha) to planar phospholipid bilayers”, J Biol Chem 265:4923 (1990).

Blankenberg, F. G., Katsikis, P. D., et al., “In vivo detection and imaging of phosphatidyl-serine expression during programmed cell death”, Proc Natl Acad Sci USA 95:6349 (1998)

Blankenberg, F. G., Katsikis, P. D., et al., “Imaging of apoptosis (programmed cell death) with Tc-99m annexin V”, Journal of Nuclear Medicine 40 (1):184 (1999).

Dumont, E., Hofstra, L., et al., “Cardiomyocyte death induced by myocardial ischemia and reperfusion—Measurement with recombinant human annexin-V in a mouse model”, Circulation 102:1564 (2000).

Dumont, E. A., Reutelingsperger, C. P., et al., “Real-time imaging of apoptotic cell-membrane changes at the single-cell level in the beating murine heart”, Nat Med 7:1352 (2001).

Fadok, V. A., et al., “Exposure of phosphatidylserine on the surface of apoptotic lymphocytes triggers specific recognition and removal by macrophages”, J Immunol 148:2207 (1992).

Gerke, V. and Moss, S. E, “Annexins: from structure to function”, Physiol Rev 82:331 (2002).

Hofstra, L., Liem, I. H., et al., “Visualisation of cell death in vivo in patients with acute myocardial infarction”, Lancet 356:209 (2000).

Narula, J., Acio, E. R, et al., “Annexin-V imnagig for noninvasive detection of cardiac allograft rejection”, Nat Med 7:1347 (2001).

Hofstra, L., Dumont, E. A., et al., “In vivo detection of apoptosis in an intracardiac tumor”, Jama 285:1841 (2001).

Kerr, J, et al., “Apoptosis. Its significance in cancer and cancer therapy.”, Cancer 73:2013 (1994).

Lehninger, Principles of Biochemistry, third edition 2000, by David L. Nelson and Michael M. Cox on page 393.

Leist, M., et al., “Four deaths and a funeral: from caspases to alternative mechanisms”, Nat Rev Mol Cell Biol 2:589 (2001).

Mira, J. P., Dubois, T., et al., “inhibition of cytosolic phospholipase A2 by annexin V in differentiated permeabilized HL60 cells. Evidence of crucial importance of domain I type II Ca2+-binding site in the mechanism of inhibition”, J Biol Chem 272:10474 (1997).

Savill, J. and Fadok V., “Corpse clearance defines the meaning of cell death”, Nature 407:784 (2000).

Stuart MCA, CPM Reutelingsperger and PM Frederik, Binding of Annexin V to bilayers with various phospholipid compositions using glass beads in a flow cytometer. Cytometry 33.414-419 (1998).

Tait, J. F., Gibson, D., et al., “Phospholipid binding properties of human placental anticoagulant protein-I, a member of the lipocortin family”, J Biol Chem 264;7944 VandenEijnde, S. M., Boshart, L., et al., “Phosphatidylserine plasma membrane asymmetry in vivo A pancellular phenomenon which alters during apoptosis”, Cell Death and Differentiation 4 (4):311 (1997).

Van Engeland, M, Nieland, L. J. W., et al., “Annexin V-affinity assay: A review on an apoptosis detection system based on phosphatidylserine exposure”, Cytomety 31 (1):1 (1998).

Zwaal, R. F, and Schroit, A. J., “Pathophysiologic implications of membrane phospholipid asymmetry in blood cells”, Blood 89:1121 (1997).

I claim:

1. A method for determining the biologically active fraction from a mixture containing lipid-binding compounds in a liquid sample, the method comprising:
a: contacting the sample with a solid carrier crying lipids;
b: separating the solid lipid-carrying carrier from the liquid, and
c: determining the fraction bound to the separated carrier.

2. A method according to claim 1, in which said carrier is in the form of particles.

3. A method according to claim 2, in which said particles contain metal and/or metal oxides.

4. A method according to claim 3, in which said particles have or can be induced to have magnetic properties.

5. A method according to claim 3, in which said particles comprise a polymer selected from agarose, cellulose, collagen, dextran, polyacrylamide, polyvinyl, polyvinyl alcohol, polyethylene glycol, polystyrene and latex.

6. A method according to claim 1, in which said lipids comprise one or more of phosphatidylserine, cardiolipin, phosphatidic acid, phosphatidylinositol, phosphatidyl-ethanolamine, phosphatidylcholine, sphingomyelin and cholesterol and their lyso analogs.

7. A method according to claim 6, in which said lipids comprise phosphatidylserine.

8. A method according to claim 1, in which the lipid-binding compounds are polypeptides.

9. A method according to claim 8, in which the lipid-binding compounds comprise a member of the Annexin family, and said contacting step (a) is carried out in the presence of divalent cations.

10. A method according to 1, in which at least one of the lipid-binding compounds is labeled with a radioactive tracer, a fluorescent probe or an antibody or part thereof.

11. A kit for performing the method of claim 1, containing lipid-coated particles, said particles having or being inducible to have magnetic properties.

12. A kit according to claim 11, further containing a magnet as a separator means.

13. A method for determining the presence of compounds in a biological sample that neutralize the biological activity of lipid-binding proteins, the method comprising;
a: mixing the biological sample with a known amount of a lipid-binding protein; and
b: contacting the lipid-binding protein, before, during or after step a, with a solid carrier carrying lipids; and
c: separating the solid carrier carrying lipids with a separator from the liquid; and
d: determining the fraction of lipid-binding protein bound to the separated carrier.

14. A method according to claim 13, in which said carrier is in the form of particles.

15. A method according to claim 14, in which said particles contain metal and/or metal oxides.

16. A method according to claim 15, in which said particles have or can be induced to have magnetic properties.

17. A method according to claim 13, in which said lipids comprise phosphatidylserine.

18. A method according to claim 13, in which a the lipid-binding compounds comprise a member of the Annexin family, and said contacting step (b) is carried out in the presence of divalent cations.

19. A method according to claim 13, in which at least one of the lipid-binding compounds is labeled with a radioactive tracer, a fluorescent probe or an antibody or part thereof.

20. A kit for performing the method of claim 13 containing
lipid-coated particles;
a lipid-binding protein.

21. A kit according to claim 20, father containing divalent cations.
